(19) United States
(12) Patent Application Publication
(10) Pub. No.: US 2017/0367754 A1
(43) Pub. Date: Dec. 28, 2017

(54) MEDICAL TREATMENT DEVICE, METHOD FOR OPERATING MEDICAL TREATMENT DEVICE, AND TREATMENT METHOD

(71) Applicant: OLYMPUS CORPORATION, Tokyo (JP)

(72) Inventor: Masato NARISAWA, Tokyo (JP)

(73) Assignee: OLYMPUS CORPORATION, Tokyo (JP)

(21) Appl. No.: 15/683,084

(22) Filed: Aug. 22, 2017

Related U.S. Application Data
(63) Continuation of application No. PCT/JP2015/055978, filed on Feb. 27, 2015.

(51) Int. Cl. A61B 18/14; A61N 7/02; A61B 18/00

(52) U.S. Cl. CPC A61B 18/1445; A61N 7/02; A61B 2018/00095

(57) ABSTRACT

A medical treatment device includes: a pair of holding members configured to grasp a target part to be connected in a body tissue; an energy application portion provided on at least one holding member of the pair of holding members, the energy application portion being configured to contact the target part when the target part is grasped by the pair of holding members to apply energy to the target part; and a processor including hardware. The processor is configured to cause the energy application portion to: apply high-frequency energy to the target part for a first period; apply ultrasound energy to the target part for a second period subsequent to the first period; and apply heat energy to the target part for a third period subsequent to the second period.

### CROSS REFERENCES TO RELATED APPLICATIONS

This application is a continuation of PCT international application Ser. No. PCT/JP2015/055978, filed on Feb. 27, 2015 which designates the United States, incorporated herein by reference.

### BACKGROUND

### 1. Technical Field

The disclosure relates to a medical treatment device, a method for operating the medical treatment device, and a treatment method.

### 2. Related Art

In recent years, developments of medical treatment devices have been accelerated with which energy is applied to a target to be connected (hereinafter described as a target part) in a body tissue to connect the target part. Such medical treatment devices do not leave a physical object such as a stapler in a body, and therefore have an advantage that there are less adverse effects on a human body. On the other hand, connection strength thereof is weaker than that of the stapler or the like, and there are some target parts which cannot be connected depending on thickness thereof. Therefore, enhancement in connection strength has been demanded.

Extracellular matrix (such as collagen or elastin) of a body tissue is constituted by a fibrous tissue. Accordingly, the connection strength is considered to be enhanced by extracting extracellular matrix from a target part and closely tangling the extracellular matrix when connecting the target part.

A medical treatment device focused on the extracellular matrix and aimed at enhancement of the connection strength has been proposed (for example, see JP 2012-239899 A).

The medical treatment device disclosed in JP 2012-239899 A grasps a target part with a pair of jaws, applies mechanical vibration to the target part (applies ultrasound energy to the target part) via the pair of jaws, thereby enhancing extraction and mixing of the extracellular matrix.

### SUMMARY

In some embodiments, a medical treatment device includes: a pair of holding members configured to grasp a target part to be connected in a body tissue; an energy application portion provided on at least one holding member of the pair of holding members, the energy application portion being configured to contact the target part when the target part is grasped by the pair of holding members to apply energy to the target part; and a processor including hardware. The processor is configured to cause the energy application portion to: apply high-frequency energy to the target part for a first period; apply ultrasound energy to the target part for a second period subsequent to the first period; and apply heat energy to the target part for a third period subsequent to the second period.

In some embodiments, a method for operating a medical treatment device includes: after a target part to be connected in a body tissue is grasped by a pair of holding members, applying high-frequency energy to the target part from at least one holding member of the pair of holding members, for a first period; applying ultrasound energy to the target part from at least one holding member of the pair of holding members, for a second period subsequent to the first period; and applying heat energy to the target part from at least one holding member of the pair of holding members, for a third period subsequent to the second period.

In some embodiments, a treatment method includes: grasping, by a pair of holding members, a target part to be connected in a body tissue; applying high-frequency energy to the target part from at least one holding member of the pair of holding members, for a first period; applying ultrasound energy to the target part from at least one holding member of the pair of holding members, for a second period subsequent to the first period; and applying heat energy to the target part from at least one holding member of the pair of holding members, for a third period subsequent to the second period.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a diagram schematically illustrating a medical treatment device according to a first embodiment of the present invention;

FIG. 2 is a block diagram illustrating a configuration of a control device illustrated in FIG. 1;

FIG. 3 is a flowchart illustrating connection control performed by the control device illustrated in FIG. 2;

FIG. 4 is a graph illustrating a behavior of impedance of a target part calculated at Step S4 or later illustrated in FIG. 3;

FIG. 5 is a graph illustrating a behavior of impedance of an ultrasound transducer calculated at Step S7 or later illustrated in FIG. 3;

FIG. 6 is a time chart illustrating types of energy applied, and compression loads applied on a target part, for first to third periods in the connection control illustrated in FIG. 3;

FIG. 7 is a chart illustrating a modification of the first embodiment of the present invention;

FIG. 8 is a block diagram illustrating a configuration of a medical treatment device according to a second embodiment of the present invention;

FIG. 9 is a diagram explaining a function of a lock mechanism illustrated in FIG. 8; and

FIG. 10 is a flowchart illustrating connection control performed by a control device illustrated in FIG. 8.

### DETAILED DESCRIPTION

Exemplary embodiments of the present invention will be described below with reference to the drawings. The present invention is not limited by the embodiments described below. The same reference signs are used to designate the same elements throughout the drawings.

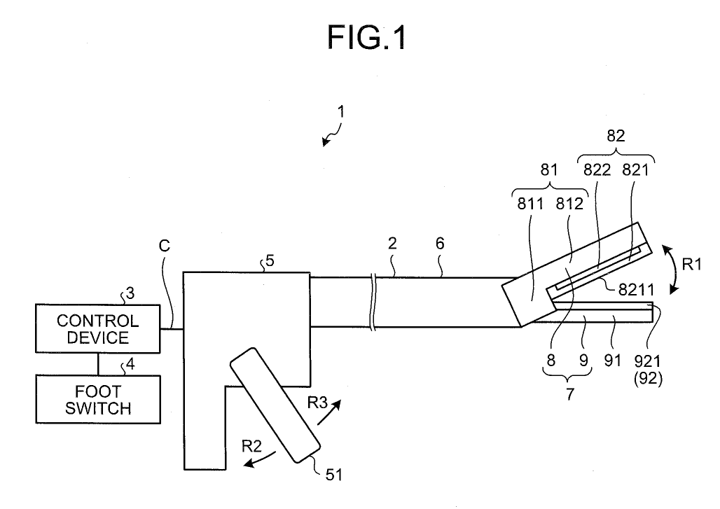

### First Embodiment

[Schematic Configuration of Medical Treatment Device] FIG. 1 is a diagram schematically illustrating a medical treatment device 1 according to a first embodiment of the present invention.

The medical treatment device 1 applies energy (high-frequency energy, ultrasound energy, and heat energy) to a site as a target (hereinafter described as a target part) of a treatment (connection or anastomosis) in a body tissue to treat the target part. As illustrated in FIG. 1, the medical treatment device 1 includes a treatment tool 2, a control device 3, and a foot switch 4.

Configuration of Treatment Tool

The treatment tool 2 is, for example, a surgical medical treatment tool of a linear type, used for treating a target part through an abdominal wall. As illustrated in FIG. 1, the treatment tool 2 includes a handle 5, a shaft 6, and a grasping portion 7.

The handle 5 is a portion held by an operator. As illustrated in FIG. 1, the handle 5 is provided with an operation knob 51.

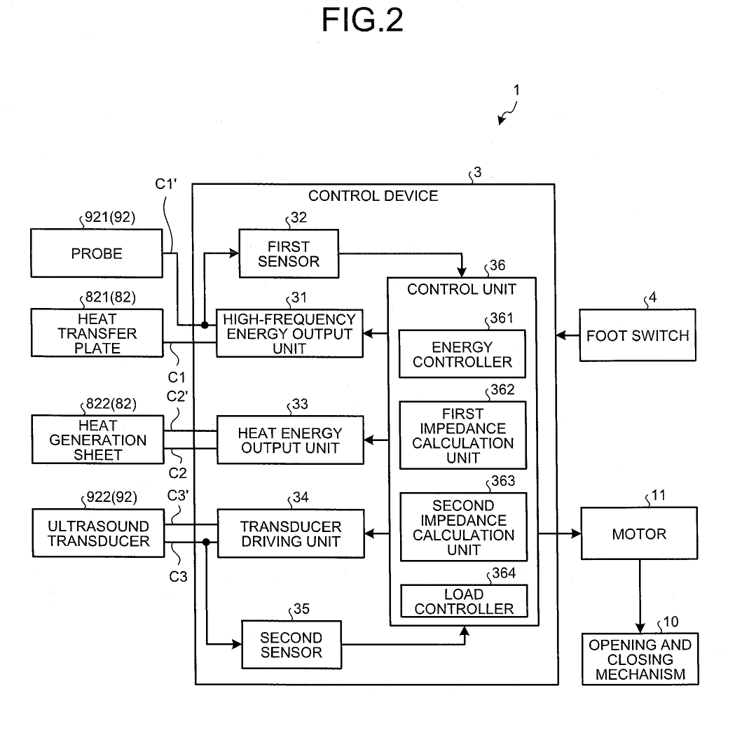

The shaft 6 has substantially a cylindrical shape, and one end thereof is connected to the handle 5 (FIG. 1). The grasping portion 7 is attached to another end of the shaft 6. An opening and closing mechanism 10 (see FIG. 2) is provided inside the shaft 6. The opening and closing mechanism 10 opens and closes first and second holding members 8 and 9 (FIG. 1) constituting the grasping portion 7 in accordance with an operation of the operation knob 51 by the operator. A motor 11 (see FIG. 2) is provided inside the handle 5. The motor 11 is connected to the opening and closing mechanism 10, and when the first and second holding members 8 and 9 grasp a target part, the motor 11 increases a compression load to be applied to the target part from the first and second holding members 8 and 9 by causing the opening and closing mechanism 10 to operate under control of the control device 3. Furthermore, an electric cable C (FIG. 1) connected to the control device 3 is arranged inside the shaft 6 from one end to the other end thereof via the handle 5.

Configuration of Grasping Portion

The grasping portion 7 is a portion for grasping a target part and treating the target part. As illustrated in FIG. 1, the grasping portion 7 includes the first holding member 8 and the second holding member 9.

The first and second holding members 8 and 9 are configured to be capable of opening and closing (capable of grasping the target part) in an arrow R1 (FIG. 1) direction in accordance with an operation of the operation knob 51 by the operator.

Specifically, as illustrated in FIG. 1, the first holding member 8 is axially supported in a rotatable manner at the other end of the shaft 6. On the other hand, the second holding member 9 is fixed at the other end of the shaft 6. In other words, in the first embodiment, the first holding member 8 is configured to be capable of opening and closing with respect to the second holding member 9 in accordance with the operation of the operation knob 51 by the operator. For example, when the operation knob 51 is moved in an arrow R2 (FIG. 1) direction, the first holding member 8 rotates in a direction close to the second holding member 9. Alternatively, when the operation knob 51 is moved in an arrow R3 (FIG. 1) direction, which is opposite to the arrow R2 direction, the first holding member 8 rotates in a direction away from the second holding member 9.

The first holding member 8 is arranged, in FIG. 1, on a side above the second holding member 9. The first holding member 8 includes a first jaw 81 and a first energy application portion 82.

As illustrated in FIG. 1, the first jaw 81 includes an axially supported portion 811 axially supported at the other end of the shaft 6 and a support plate 812 connected to the axially supported portion 811, and opens and closes in the arrow R1 direction in accordance with an operation of the operation knob 51 by the operator.

The first energy application portion 82 applies high-frequency energy and heat energy to the target part under control of the control device 3. As illustrated in FIG. 1, the first energy application portion 82 includes a heat transfer plate 821 and a heat generation sheet 822. The heat generation sheet 822 and the heat transfer plate 821 are stacked in this order on a plate surface of the support plate 812 opposite to the second holding member 9.

The heat transfer plate 821 is constituted, for example, by a thin copper plate.

When a target part is grasped by the first and second holding members 8 and 9, a plate surface on a lower side of the heat transfer plate 821 in FIG. 1 functions as a treatment surface 8211 which contacts the target part.

Then, the heat transfer plate 821 transfers heat from the heat generation sheet 822 to the target part from the treatment surface 8211 (applies heat energy to the target part). A high-frequency lead wire C1 (see FIG. 2) constituting the electric cable C is connected to the heat transfer plate 821, the control device 3 supplies high-frequency power to between the heat transfer plate 821 and a probe 921 described later via the high-frequency lead wire C1 and a high-frequency lead wire C1′ (see FIG. 2), and thereby the heat transfer plate 821 applies high-frequency energy to the target part.

The heat generation sheet 822 functions as a sheet-type heater. Although specific illustration is omitted, the heat generation sheet 822 has a configuration in which an electric resistance pattern is formed by deposition or the like on a sheet-shaped substrate constituted by an insulating material such as polyimide.

The electric resistance pattern is formed along a U-shape following a peripheral shape of the heat generation sheet 822, and heat-generation lead wires C2 and C2′ (see FIG. 2) constituting the electric cable C are connected to both ends thereof. Then, the control device 3 applies a voltage to (applies current to) the electric resistance pattern via the heat-generation lead wires C2 and C2′ to cause the electric resistance pattern to generate heat.

Although illustration is omitted in FIG. 1, an adhesive sheet is interposed between the heat transfer plate 821 and the heat generation sheet 822 for adhering the heat transfer plate 821 and the heat generation sheet 822 together. The adhesive sheet has high thermal conductivity, withstands high temperatures, and has adhesiveness. The adhesive sheet is formed, for example, by mixing an epoxy resin with ceramics having high thermal conductivity such as alumina, aluminum nitride, or the like.

As illustrated in FIG. 1, the second holding member 9 includes a second jaw 91 and a second energy application portion 92.

The second jaw 91 is fixed at the other end of the shaft 6, and has a shape extending along an axial direction of the shaft 6.

The second energy application portion 92 applies ultrasound energy to the target part under control of the control device 3. The second energy application portion 92 includes the probe 921 (FIG. 1) and an ultrasound transducer 922 (see FIG. 2).

The probe 921 is a column constituted by a conductive material, and extending along the axial direction of the shaft 6. As illustrated in FIG. 1, the probe 921 is inserted into the shaft 6 while one end (in FIG. 1, a right end) thereof is exposed outside, and the ultrasound transducer 922 is attached to another end thereof. When a target part is grasped by the first and second holding members 8 and 9, the probe 921 contacts the target part and transmits ultrasound vibration generated by the ultrasound transducer 922 to the target part (applies ultrasound energy to the target part).

The ultrasound transducer 922 is configured, for example, by a piezoelectric transducer including a piezoelectric element which extends and contracts by application of an alternating-current voltage. Ultrasound lead wires C3 and C3′ (see FIG. 2) constituting the electric cable C are connected to the ultrasound transducer 922, an alternating-current voltage is applied to the ultrasound transducer 922 under control of the control device 3, and thereby the ultrasound transducer 922 generates ultrasound vibration.

Although specific illustration is omitted, a vibration enhancing member such as a horn for enhancing the ultrasound vibration generated by the ultrasound transducer 922 is interposed between the ultrasound transducer 922 and the probe 921.

Here, as a configuration of the second energy application portion 92, the probe 921 may vibrate longitudinally (vibrate in an axial direction of the probe 921), or the probe 921 may vibrate laterally (vibrate in a radial direction of the probe 921).

Configurations of Control Device and Foot Switch

FIG. 2 is a block diagram illustrating a configuration of the control device 3.

In FIG. 2, major parts of the present invention are mainly illustrated as the configuration of the control device 3.

The foot switch 4 is operated by a foot of the operator, and outputs an operation signal to the control device 3 in accordance with the operation (ON). Then, the control device 3 starts connection control described later in accordance with the operation signal.

Examples of means for starting the connection control include, but are not limited to, the foot switch 4. A switch operated by a hand, or the like may be employed.

The control device 3 integrally controls operations of the treatment tool 2. As illustrated in FIG. 2, the control device 3 includes a high-frequency energy output unit 31, a first sensor 32, a heat energy output unit 33, a transducer driving unit 34, a second sensor 35, and a control unit (processor) 36.

The high-frequency energy output unit 31 supplies high-frequency power between the heat transfer plate 821 and the probe 921 via the high-frequency lead wires C1 and C1′ under control of the control unit 36.

The first sensor 32 detects a voltage and a current supplied to the heat transfer plate 821 and the probe 921 from the high-frequency energy output unit 31. Then, the first sensor 32 outputs a signal in accordance with the detected voltage and current to the control unit 36.

The heat energy output unit 33 applies a voltage to (applies current to) the heat generation sheet 822 via the heat-generation lead wires C2 and C2′ under control of the control unit 36.

The transducer driving unit 34 applies an alternating-current voltage to the ultrasound transducer 922 via the ultrasound lead wires C3 and C3′ under control of the control unit 36.

The second sensor 35 detects a voltage and a current applied to the ultrasound transducer 922 from the transducer driving unit 34. Then, the second sensor 35 outputs a signal in accordance with the detected voltage and current to the control unit 36.

The control unit 36 is configured to include a central processing unit (CPU) and the like, and executes the connection control in accordance with a predetermined control program when the foot switch 4 is turned ON. As illustrated in FIG. 2, the control unit 36 includes an energy controller 361, a first impedance calculation unit 362, a second impedance calculation unit 363, and a load controller 364.

The energy controller 361 controls operations of the high-frequency energy output unit 31, the heat energy output unit 33, and the transducer driving unit 34 in accordance with the operation signal from the foot switch 4, and impedance of a target part and impedance of the ultrasound transducer 922 calculated by the first and second impedance calculation units 362 and 363, respectively. In other words, the energy controller 361 controls timing for applying high-frequency energy, ultrasound energy, and heat energy to the target part from the first and second energy application portions 82 and 92.

The first impedance calculation unit 362 calculates impedance of the target part when the high-frequency energy is applied to the target part based on the voltage and the current detected by the first sensor 32.

The second impedance calculation unit 363 calculates impedance of the ultrasound transducer 922 when the ultrasound energy is applied to the target part based on the voltage and the current detected by the second sensor 35.

Based on the impedance of the ultrasound transducer 922 calculated by the second impedance calculation unit 363, the load controller 364 causes the motor 11 to operate, and increases a compression load (force for grasping the target part by the first and second holding members 8 and 9) applied to the target part from the first and second holding members 8 and 9.

Operations of Medical Treatment Device

Next, operations of the medical treatment device 1 will be described.

Here, reference will be made mainly to connection control by the control device 3 as the operations of the medical treatment device 1.

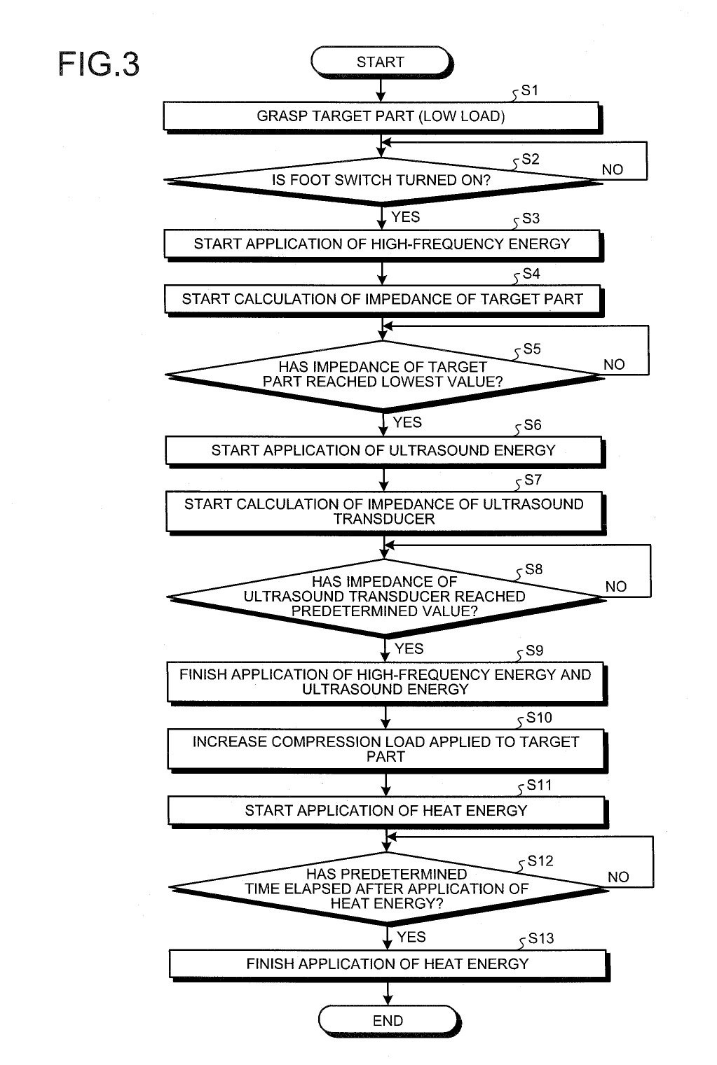

FIG. 3 is a flowchart illustrating the connection control performed by the control device 3.

The operator holds the treatment tool 2, and inserts a distal end portion (the grasping portion 7 and a part of the shaft 6) of the treatment tool 2 into a peritoneal cavity through an abdominal wall, for example, by using a trocar. Then, the operator operates the operation knob 51, opens and closes the first and second holding members 8 and 9, and grasps the target part by the first and second holding members 8 and 9 (Step S1: grasping step).

Then, the operator performs an (ON) operation of the foot switch 4 to cause the control device 3 to start the connection control.

When the operation signal from the foot switch 4 is input (the foot switch 4 is turned ON) (Step S2: Yes), the energy controller 361 drives the high-frequency energy output unit 31 to start supplying high-frequency power to the heat transfer plate 821 and the probe 921 from the high-frequency energy output unit 31 (start application of high-frequency energy to the target part) (Step S3: a first application step).

After Step S3, the first impedance calculation unit 362 starts calculating impedance of the target part based on the voltage and the current detected by the first sensor 32 (Step S4).

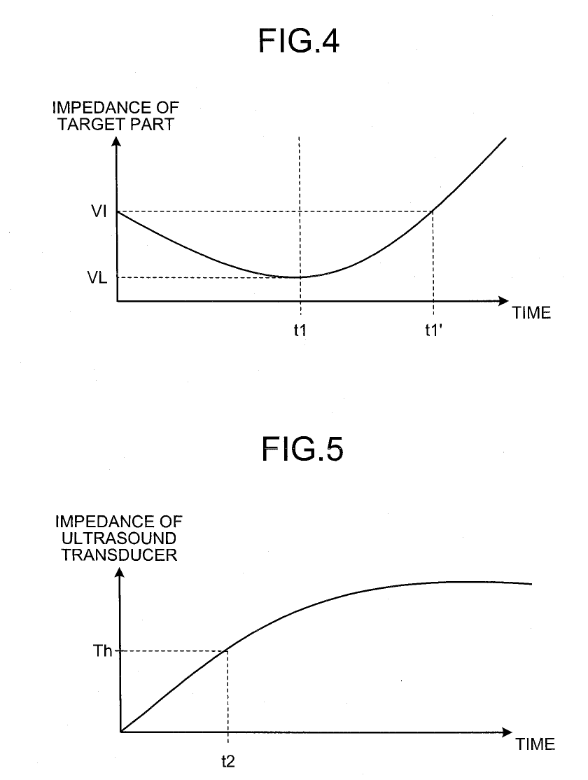

FIG. 4 is a graph illustrating a behavior of the impedance of the target part calculated at Step S4 or later.

When high-frequency energy is applied to the target part, the impedance of the target part exhibits the behavior illustrated in FIG. 4.

As illustrated in FIG. 4, the impedance of the target part gradually decreases in an initial time slot (from the start of application of the high-frequency energy to time t1) after applying the high-frequency energy. This is because cell membranes in the target part are disrupted by the applied high-frequency energy and extracellular matrix is extracted from the target part. In other words, the initial time slot is a time slot in which the extracellular matrix is extracted from the target part, so that the viscosity of the target part is decreasing (the target part is in the process of softening).

After time t1 when the impedance of the target part reaches a lowest value VL, the impedance of the target part gradually increases as illustrated in FIG. 4. This is because the applied high-frequency energy causes Joule heat to act on the target part to cause the target part itself to generate heat, and thereby moisture in the target part decreases (evaporates). In other words, time slots after time t1 are time slots in which the extracellular matrix is less and less extracted from the target part and the moisture in the target part evaporates by the generated heat, so that the viscosity of the target part is increasing (the target part is in the process of coagulation).

After Step S4, the energy controller 361 constantly monitors whether the impedance of the target part calculated by the first impedance calculation unit 362 has reached the lowest value VL (Step S5).

When it is determined that the impedance of the target part has reached the lowest value VL (Step S5: Yes), the energy controller 361 drives the transducer driving unit 34 to start application of an alternating-current voltage to the ultrasound transducer 922 from the transducer driving unit 34 (start application of ultrasound energy to the target part) (Step S6: a second application step).

After Step S6, the second impedance calculation unit 363 starts calculating impedance of the ultrasound transducer 922 based on the voltage and the current detected by the second sensor 35 (Step S7).

FIG. 5 is a graph illustrating a behavior of the impedance of the ultrasound transducer 922 calculated at Step S7 or later.

When ultrasound energy is applied to the target part, the impedance of the ultrasound transducer 922 exhibits the behavior illustrated in FIG. 5.

The impedance of the ultrasound transducer 922 increases in accordance with a load on the probe 921 when the first and second holding members 8 and 9 grasp the target part.

As described above, by the applied high-frequency energy and ultrasound energy, the moisture in the target part evaporates and thus the viscosity thereof increases. Accordingly, after time t1, the load on the probe 921 gradually increases since coagulation in the target part proceeds. In other words, the impedance of the ultrasound transducer 922 gradually increases as illustrated in FIG. 5.

After Step S7, the energy controller 361 constantly monitors whether the impedance of the ultrasound transducer 922 calculated by the second impedance calculation unit 363 has reached a predetermined value Th (FIG. 5) (Step S8).

When it is determined that the impedance of the ultrasound transducer 922 has reached the predetermined value Th (Step S8: Yes), the energy controller 361 stops driving the high-frequency energy output unit 31 and the transducer driving unit 34 (finishes application of the high-frequency energy and the ultrasound energy to the target part) (Step S9).

After Step S9, the load controller 364 causes the motor 11 to operate to increase a compression load to be applied to the target part from the first and second holding members 8 and 9 (Step S10).

After Step S10, the energy controller 361 drives the heat energy output unit 33 to start application of a voltage to (application of current to) the heat generation sheet 822 from the heat energy output unit 33 (i.e., start application of heat energy to the target part) (Step S11: a third application step).

After Step S11, the energy controller 361 constantly monitors whether a predetermined time has elapsed after the application of the heat energy in Step S11 (Step S12).

When it is determined that the predetermined time has elapsed (Step S12: Yes), the energy controller 361 stops driving the heat energy output unit 33 (finishes application of the heat energy to the target part) (Step S13).

Through the above treatments, the target part is connected.

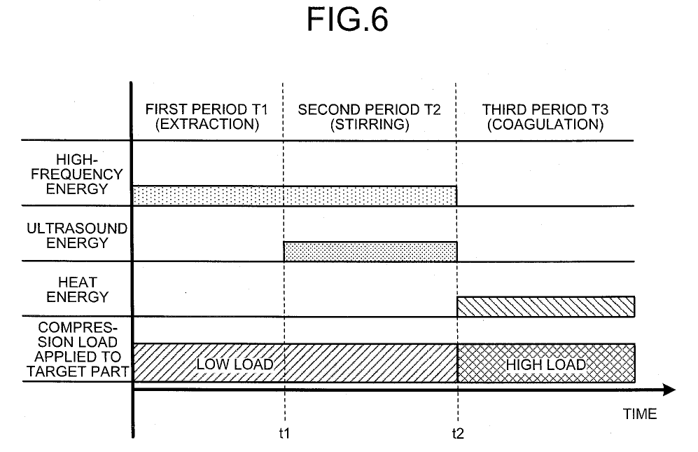

FIG. 6 is a time chart illustrating types of energy applied, and compression loads applied on the target part, in first to third periods in the connection control illustrated in FIG. 3.

Timing for applying each of high-frequency energy, ultrasound energy, and heat energy, and timing for changing a compression load to be applied to the target part are outlined as illustrated in FIG. 6.

In other words, during the first period T1 from the foot switch 4 being turned ON to time t1, only the high-frequency energy is applied to the target part as illustrated in FIG. 6. In the first period T1, a compression load applied to the target part from the first and second holding members 8 and 9 is relatively low (for example, about 0.2 MPa).

In the second period T2 which is a period from time t1 to time t2, both of the high-frequency energy and the ultrasound energy are applied to the target part as illustrated in FIG. 6. In the second period T2, a compression load applied to the target part from the first and second holding members 8 and 9 is the same as that in the first period T1.

During the third period T3 from time t2 until the predetermined time has elapsed, which is determined in Step S12, only the heat energy is applied to the target part. In the third period T3, a compression load applied to the target part from the first and second holding members 8 and 9 is higher than those in the first and second periods T1 and T2.

Here, as described above, in the medical treatment device 1 according to the first embodiment, compression loads applied to the target part from the first and second holding members 8 and 9 when the target part is grasped by the first and second holding members 8 and 9 are adjusted to be higher in the third period T3 than in the first and second periods T1 and T2.

In other words, by adjusting the compression load to be applied to the target part to be higher at coagulation of the extracellular matrix (in the third period T3), tight connection can be achieved. In addition, by adjusting the compression loads applied to the target part to be lower at extraction and stirring of the extracellular matrix (in the first and second periods T1 and T2), the extracted extracellular matrix can be prevented from flowing out from between the first and second holding members 8 and 9. In addition, although the higher the compression load applied to the target part at stirring of the extracellular matrix, the more the ultrasound energy (ultrasound vibration) is transmitted to not the target part but the first jaw 81, by adjusting the compression load to be lower as in the first embodiment, the ultrasound energy (ultrasound vibration) can be efficiently transmitted to the target part.

In the medical treatment device 1 according to the first embodiment described above, after a target part is grasped by the first and second holding members 8 and 9, high-frequency energy is applied for the first period T1, ultrasound energy is applied for the second period T2 subsequent to the first period T1, and heat energy is applied for the third period T3 subsequent to the second period T2, to the target part. In other words, cell membranes in the target part are disrupted by the high-frequency energy applied in the first period T1 to extract extracellular matrix, the extracellular matrix is stirred and closely tangled by the ultrasound energy applied in the second period T2, and the extracellular matrix is coagulated by the heat energy applied in the third period T3.

Therefore, according to the medical treatment device 1 according to the first embodiment, effects can be obtained with which three processes of extraction, stirring, and coagulation of extracellular matrix required to connect a target part can be executed appropriately, and connection strength of the target part can be enhanced.

In the medical treatment device 1 according to the first embodiment, the second period T2 is started and the ultrasound energy is applied to the target part when impedance of the target part reaches the lowest value VL.

Accordingly, it is possible to appropriately set the first period T1 during which the high-frequency energy is applied to the target part to execute a stirring process after extracting a sufficient amount of extracellular matrix from the target part, and as a result, the connection strength of the target part can be further enhanced.

In the medical treatment device 1 according to the first embodiment, the third period T3 is started and the heat energy is applied to the target part when impedance of the ultrasound transducer 922 reaches the predetermined value Th.

Accordingly, it is possible to appropriately set the second period T2 during which the ultrasound energy is applied to the target part to execute a coagulation process after sufficiently stirring the extracellular matrix, and as a result, the connection strength of the target part can be further enhanced.

### Modification of First Embodiment

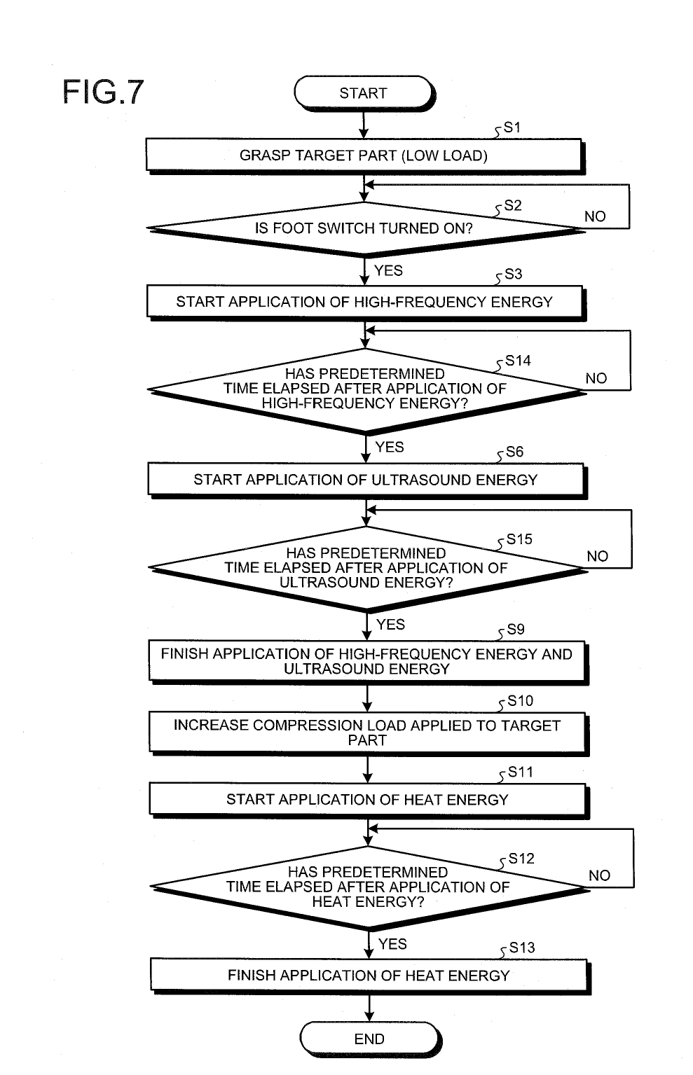

FIG. 7 is a chart illustrating a modification of the first embodiment of the present invention. Specifically, FIG. 7 is a flowchart illustrating connection control in the modification.

In the first embodiment, the application of the ultrasound energy to the target part is started based on the impedance of the target part and the application of the heat energy to the target part is started based on the impedance of the ultrasound transducer 922 (the compression load to be applied to the target part is increased). Alternatively, the application of each energy described above may be started when a predetermined time has elapsed as in the modification.

In other words, in the modification, the first and second sensors 32 and 35 as well as the first and second impedance calculation units 362 and 363 are omitted. In the connection control in the modification, Steps S4, S5, S7, and S8 are omitted, and Steps S14 and S15 are added, as illustrated in FIG. 7, to the connection control (FIG. 3) described in the first embodiment. Steps S4, S5, S7, and S8 relate to calculation of impedance of each of the target part and the ultrasound transducer 922.

Step S14 is executed after Step S3.

Specifically, the energy controller 361 constantly monitors in Step S14 whether a predetermined time has elapsed after the application of the high-frequency energy in Step S3.

The predetermined time used herein is time set as follows.

In other words, each of Steps S3 to S5 is executed for a plurality of other body tissues in advance. Then, time taken for impedance of the target part to reach the lowest value VL from the start of the high-frequency energy application is acquired for each body tissue, and an average value of the acquired time is set as the predetermined time to be determined in Step S14.

When it is determined that the predetermined time has elapsed after the application of the high-frequency energy (Step S14: Yes), the control device 3 proceeds to Step S6.

Step S15 is executed after Step S6.

Specifically, the energy controller 361 constantly monitors in Step S15 whether a predetermined time has elapsed after the application of the ultrasound energy in Step S6.

The predetermined time used herein is time set as follows.

In other words, each of Steps S3 to S8 is executed for a plurality of other body tissues in advance. Then, time taken for impedance of the ultrasound transducer 922 to reach the predetermined value Th from the start of the ultrasound energy application is acquired for each body tissue, and an average value of the acquired time is set as the predetermined time to be determined in Step S15.

When it is determined that the predetermined time has elapsed after the application of the ultrasound energy (Step S15: Yes), the control device 3 proceeds to Step S9.

According to the modification, similar effects to those in the first embodiment can be obtained, and in addition, the configuration can be simplified by omitting the first and second sensors 32 and 35, as well as the first and second impedance calculation units 362 and 363.

### Second Embodiment

Next, a second embodiment of the present invention will be described.

In the following description, the same reference signs are used to designate the same elements as those in the first embodiment, and detailed descriptions thereof will be omitted or simplified.

In the medical treatment device 1 according to the first embodiment, the motor 11 and the load controller 364 are employed to automatically increase a compression load to be applied to the target part at the start of the application of the heat energy.

On the contrary, in a medical treatment device according to the second embodiment, a compression load to be applied to a target part at the start of application of heat energy is increased by manual operation.

Here, reference will be made to connection control and the configuration of the medical treatment device according to the second embodiment.

Configuration of Medical Treatment Device

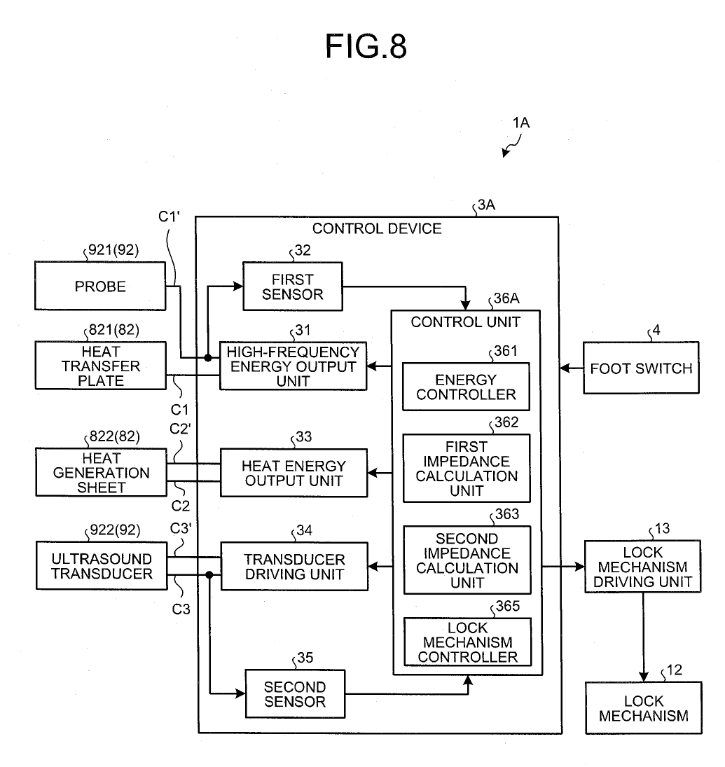

FIG. 8 is a block diagram illustrating a configuration of a medical treatment device 1A according to the second embodiment of the present invention.

In the medical treatment device 1A according to the second embodiment, the motor 11 and the load controller 364 are omitted as illustrated in FIG. 8, in comparison to the medical treatment device 1 (FIGS. 1 and 2) described in the first embodiment. In addition, in the medical treatment device 1A, a lock mechanism 12 and a lock mechanism driving unit 13 are added and a part of the functions of a control unit 36 is changed, in comparison to the medical treatment device 1 described in the first embodiment.

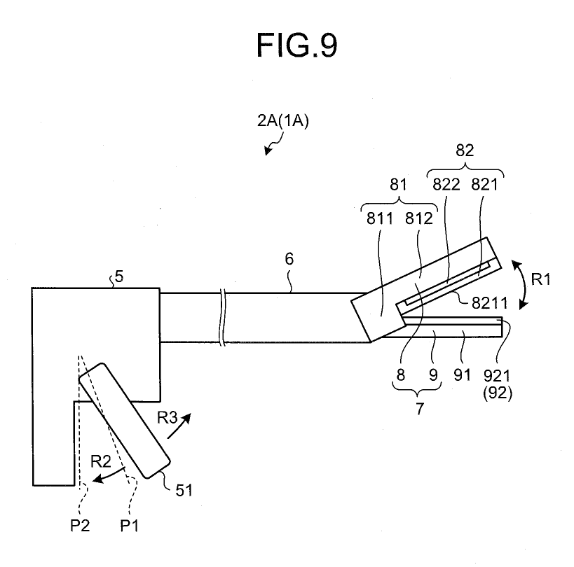

FIG. 9 is a diagram explaining a function of the lock mechanism 12. Specifically, FIG. 9 is a diagram illustrating a treatment tool 2A according to the second embodiment.

The lock mechanism 12 is provided inside a handle 5 and is configured to switch an operation knob 51 to a permissive state or to a restrictive state.

Specifically, the lock mechanism 12 mechanically connects (locks) the operation knob 51 or an opening and closing mechanism 10 in the restrictive state, thereby restricting movement of the operation knob 51 from a first position P1 (FIG. 9) to a second position P2 (FIG. 9). In addition, the lock mechanism 12 mechanically disconnects (unlocks) the operation knob 51 or the opening and closing mechanism 10 in the permissive state, thereby permitting movement of the operation knob 51.

Here, the first position P1 is a position described below.

When the operation knob 51 is moved to the first position P1 from an initial position (a position of the operation knob 51 illustrated in FIG. 9), a first holding member 8 rotates in a direction close to a second holding member 9, thereby applying a relatively low compression load (a first compression load (for example, about 0.2 MPa)) to a target part grasped between the first holding member 8 and the second holding member 9. In other words, the first position P1 is a position where the first compression load is applied to the target part.

In addition, the second position P2 is a position described below.

When the operation knob 51 is moved to the second position P2 from the first position P1, the first holding member 8 rotates in a direction closer to the second holding member 9, thereby applying a second compression load higher than the first compression load to the target part grasped between the first holding member 8 and the second holding member 9. In other words, the second position P2 is a position where the second compression load is applied to the target part.

In the second embodiment, the lock mechanism 12 is constantly biased by a bias member, such as a spring, so as to mechanically connect (lock) the operation knob 51 or the opening and closing mechanism 10.

The lock mechanism driving unit 13 is provided inside the handle 5, and is configured to switch the operation knob 51 to the permissive state from the restrictive state by causing the lock mechanism 12 to operate against bias force of the bias member such as a spring under control of a control device 3A (control unit 36A).

As illustrated in FIG. 8, in the control unit 36A, the load controller 364 is omitted and a lock mechanism controller 365 is added, in comparison to the control unit 36 (FIG. 2) described in the first embodiment.

The lock mechanism controller 365 drives the lock mechanism driving unit 13 based on impedance of an ultrasound transducer 922 calculated by a second impedance calculation unit 363 to switch the operation knob 51 to the permissive state from the restrictive state.

Connection Control

Next, connection control according to the second embodiment will be described.

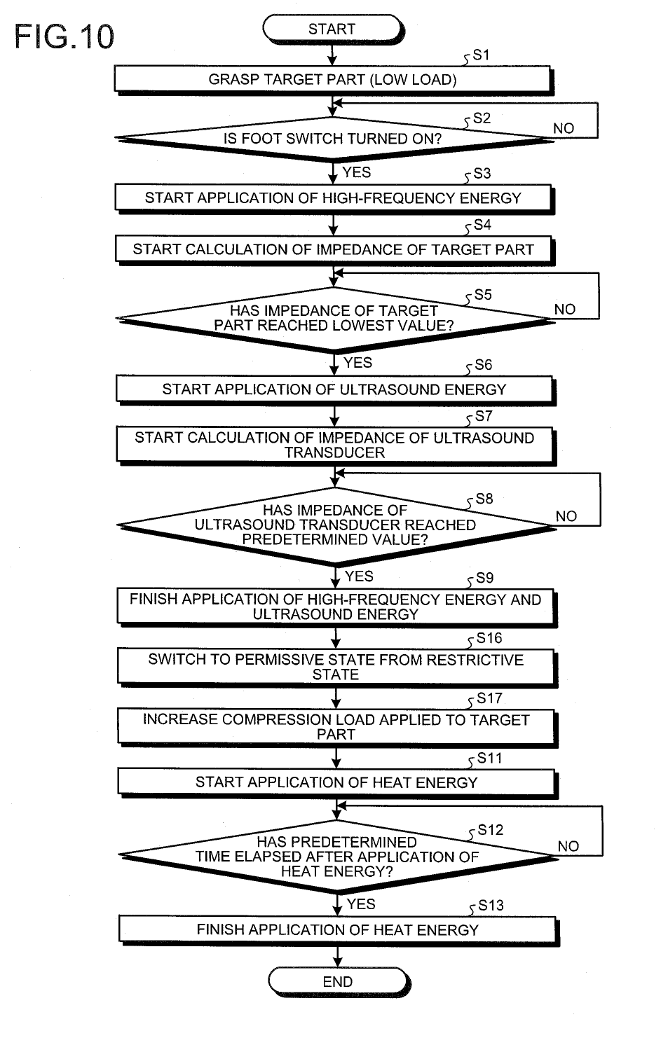

FIG. 10 is a flowchart illustrating connection control performed by the control device 3A.

As illustrated in FIG. 10, in the connection control according to the second embodiment, Step S10 relating to the operation of the motor 11 is omitted, and Steps S16 and S17 are added to the connection control (FIG. 3) described in the first embodiment.

As described above, in a state where the lock mechanism driving unit 13 is not driven, the lock mechanism 12 is biased by the bias member, such as a spring, so as to mechanically connect the operation knob 51 or the opening and closing mechanism 10 (the operation knob 51 is set to be in the restrictive state). Accordingly, in Step S1 in the second embodiment, the operator moves the operation knob 51 to the first position P1 from the initial position, and grasps the target part with the first and second holding members 8 and 9. In other words, the first compression load is applied to the target part.

Step S16 is executed after Step S9.

Specifically, the lock mechanism controller 365 drives the lock mechanism driving unit 13 in Step S16 to switch the operation knob 51 to the permissive state from the restrictive state on condition that it is determined in Step S8 that the impedance of the ultrasound transducer 922 has reached a predetermined value Th (Step S8: Yes).

After Step S16, the operator moves the operation knob 51 to the second position P2 from the first position P1 (Step S17). In other words, the second compression load higher than the first compression load is applied to the target part.

After Step S17, the control device 3A proceeds to Step S11.

According to the second embodiment described above, in addition to similar effects to those in the first embodiment, the following effect can be obtained.

In the medical treatment device 1A according to the second embodiment, the lock mechanism 12 is employed for operation by manual to increase a compression load to be applied to a target part at the start of application of heat energy.

Accordingly, the medical treatment device 1A can be manufactured inexpensively in comparison to the medical treatment device 1 using the motor 11 described in the first embodiment.

### Modification of Second Embodiment

In the second embodiment, application of ultrasound energy or heat energy may be started (the operation knob 51 may be switched from the restrictive state to the permissive state) when a predetermined time has elapsed, as in the modification (FIG. 7) of the first embodiment.

In the second embodiment, a notifying unit may be provided to notify the medical treatment device 1A that the operation knob 51 has been switched from the restrictive state to the permissive state.

Examples of the notifying unit include a light emitting diode (LED) for emitting light, a display for displaying messages, and a configuration for producing sound.

### Other Embodiments

Hereinabove, the modes for carrying out the present invention have been described. However, the present invention should not be limited exclusively to the first and second embodiments and the modifications thereof.

In the first and second embodiments and the modifications thereof, the first energy application portion 82 is provided on the first holding member 8 and the second energy application portion 92 is provided on the second holding member 9. Alternatively, an energy application portion for applying energy may be provided on only one of the first and second holding members 8 and 9 as long as high-frequency energy, ultrasound energy, and heat energy can be applied to a target part. Alternatively, each energy application portion may be provided on both of the first and second holding members 8 and 9. For example, the heat generation sheet 822 and the heat transfer plate 821 may be formed on the probe 921.

In the first and second embodiments and the modifications thereof, the high-frequency energy is applied for the first and second periods T1 and T2, the ultrasound energy is applied for the second period T2, and the heat energy is applied for the third period T3. Alternatively, two or more types of energy may be simultaneously applied in any period, as with the second period T2 in the first and second embodiments and the modifications thereof, as long as the high-frequency energy is applied at least for the first period T1, the ultrasound energy is applied at least for the second period T2, and the heat energy is applied at least for the third period T3.

In the first and second embodiments and the modifications thereof, the heat generation sheet 822 is employed to apply the heat energy to the target part. Alternatively, a plurality of heat-generating chips may be provided on the heat transfer plate 821, and current may be applied to the plurality of heat-generating chips to transfer heat of the plurality of heat-generating chips to the target part via the heat transfer plate 821 (for example, regarding the technology, see JP 2013-106909 A).

In the first and second embodiments and the modifications thereof, timing for starting application of the ultrasound energy or heat energy, or for increasing a compression load to be applied to the target part is adjusted based on impedance of the target part or the ultrasound transducer 922, or based on time. Alternatively, the above-described timing may be adjusted based on physical properties such as hardness, thickness, or temperature of the target part.

In the first and second embodiments, the application of the ultrasound energy is started when impedance of the target part has reached the lowest value VL. Alternatively, the application of the ultrasound energy may be started at any time after time t1 when the impedance of the target part reaches the lowest value VL (for example, between time t1 and time t1′ (FIG. 4) when the impedance reverts to an initial value VI (FIG. 4) at the start of the application of the high-frequency energy).

In addition, the flow of the connection control is not limited to the order of processes in flowcharts (FIGS. 3, 7, and 10) described in the first and second embodiments and the modifications thereof, and may be changed without inconsistency.

According to some embodiments, it is possible to enhance connection strength of a target part.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical treatment device comprising:
a pair of holding members configured to grasp a target part to be connected in a body tissue;
an energy application portion provided on at least one holding member of the pair of holding members, the energy application portion being configured to contact the target part when the target part is grasped by the pair of holding members to apply energy to the target part; and
a processor comprising hardware, wherein the processor is configured to cause the energy application portion to:
apply high-frequency energy to the target part for a first period;
apply ultrasound energy to the target part for a second period subsequent to the first period; and
apply heat energy to the target part for a third period subsequent to the second period.

2. The medical treatment device according to claim 1, wherein
the processor is configured to calculate impedance of the target part when the high-frequency energy is applied to the target part, wherein
after the impedance of the target part reaches a lowest value, the processor is configured to start the second period and cause the energy application portion to apply the ultrasound energy to the target part.

3. The medical treatment device according to claim 1, wherein
the energy application portion comprises an ultrasound transducer configured to apply the ultrasound energy to the target part,
the processor is configured to calculate impedance of the ultrasound transducer when the ultrasound energy is applied to the target part, and
when the impedance of the ultrasound transducer reaches a predetermined value, the processor is configured to start the third period and cause the energy application portion to apply the heat energy to the target part.

4. The medical treatment device according to claim 1, wherein
the processor is configured to switch between compression loads to be applied to the target part from the pair of holding members when the target part is grasped by the pair of holding members, wherein
the processor is configured to set a compression load in the first and second periods to be different from a compression load in the third period.

5. The medical treatment device according to claim 4, wherein
the processor is configured to set the compression load in the third period to be higher than the compression load in the first and second periods.

6. The medical treatment device according to claim 1, further comprising:
a handle configured to be relatively movable to a first position and to a second position; and
a lock mechanism configured to switch to a permissive state for permitting the handle to relatively move from the first position to the second position, or switch to a restrictive state for restricting relative movement of the handle from the first position to the second position, wherein
the processor is configured to cause the lock mechanism to operate,
when the handle is moved to the first position, the pair of holding members is configured to apply a first compression load to the target part,
when the handle is moved to the second position, the pair of holding members is configured to apply a second compression load higher than the first compression load to the target part, and
the processor is configured to set the restrictive state for the first and second periods, and set the permissive state for the third period.

7. A method for operating a medical treatment device, the method comprising:
after a target part to be connected in a body tissue is grasped by a pair of holding members, applying high-frequency energy to the target part from at least one holding member of the pair of holding members, for a first period;
applying ultrasound energy to the target part from at least one holding member of the pair of holding members, for a second period subsequent to the first period; and
applying heat energy to the target part from at least one holding member of the pair of holding members, for a third period subsequent to the second period.

8. A treatment method comprising:
grasping, by a pair of holding members, a target part to be connected in a body tissue;
applying high-frequency energy to the target part from at least one holding member of the pair of holding members, for a first period;
applying ultrasound energy to the target part from at least one holding member of the pair of holding members, for a second period subsequent to the first period; and
applying heat energy to the target part from at least one holding member of the pair of holding members, for a third period subsequent to the second period.
